(19) United States
(12) Patent Application Publication
(10) Pub. No.: US 2026/0108567 A1
(43) Pub. Date: Apr. 23, 2026

(54) FEED ADDITIVE FOR AQUACULTURE AND USE THEREOF

(71) Applicant: DABOMB PROTEIN BIOTECH CORPORATION, Tainan (TW)

(72) Inventors: YU-FEN LIU, Tainan (TW); WEI-CHIH HUANG, Tainan (TW); YU-HUNG LIN, Tainan (TW)

(21) Appl. No.: 18/923,029

(22) Filed: Oct. 22, 2024

(51) Int. Cl. A61K 35/742; A23K 10/18; A23K 10/30; A23K 50/80; A61K 35/00; A61K 35/747

(52) U.S. Cl. CPC A61K 35/742; A23K 10/18; A23K 10/30; A23K 50/80; A61K 35/747; A61K 2035/115

(57) ABSTRACT

A feed additive for aquaculture and use thereof is provided, including Bacillus amyloliquefaciens and postbiotics of Lactobacillus paracasei. A use of the feed additive for aquaculture mentioned above is further provided.

### BACKGROUND OF THE INVENTION

### Field of the Invention

The present invention relates to feed additive for aquaculture and use thereof.

### Description of the Prior Art

Large-scale farming of white shrimp requires significant amounts of fishmeal and fish oil. Fishmeal is made from processed fish and shrimp feed to provide protein and nutrients. However, this also means that large quantities of wild fish need to be caught, which leads to overfishing and ecological damage, and resulting in high costs.

In the aquaculture industry, shrimps prone to Vibrio infections, which cause early mortality, resulting in mass deaths and decreased production, leading to economic losses and increase costs.

The present invention is, therefore, arisen to obviate or at least mitigate the above-mentioned disadvantages.

### SUMMARY OF THE INVENTION

The main object of the present invention is to provide a feed additive for aquaculture and use thereof, which is utilized to improve feed conversion ratio and increase survival rate of aquatic animals.

To achieve the above and other objects, the present invention provides a feed additive for aquaculture, including: Bacillus amyloliquefaciens and postbiotics of Lactobacillus paracasei.

To achieve the above and other objects, the present invention further provides a use of the feed additive for aquaculture mentioned above for improving feed conversion ratio (FCR).

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a bar chart showing the percent weight gain analysis results of Litopenaeus vannamei fed with experimental diets according to the present invention;

FIG. 2 is a bar chart showing the feed conversion ratio analysis results of Litopenaeus vannamei fed with the experimental diets according to the present invention;

FIG. 3 is a bar chart showing the total haemocyte count analysis results of Litopenaeus vannamei fed with the experimental diets according to the present invention;

FIG. 4 is a bar chart showing the phenoloxidase activity analysis results of Litopenaeus vannamei fed with the experimental diets according to the present invention;

FIG. 5 is a bar chart showing the lysozyme activity analysis results of Litopenaeus vannamei fed with the experimental diets according to the present invention;

FIG. 6 is a bar chart showing the phagocytic activity analysis results of Litopenaeus vannamei fed with the experimental diets according to the present invention;

FIG. 7 is a bar chart showing the clearance efficiency analysis results of Litopenaeus vannamei fed with the experimental diets according to the present invention;

FIG. 8 is a bar chart showing the survival rate analysis results of Litopenaeus vannamei fed with the experimental diets according to the present invention.

### DETAILED DESCRIPTION OF THE PREFERRED EMBODIMENTS

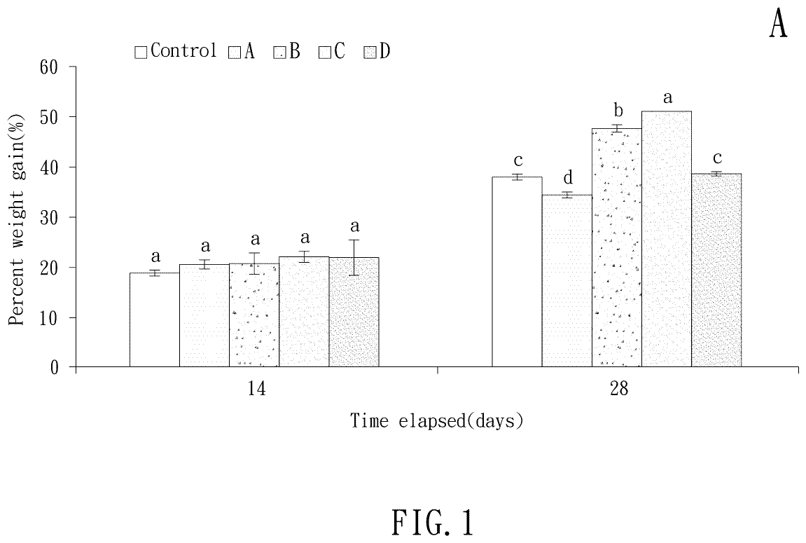

Please refer to FIGS. 1 to 8 for a preferable embodiment of the present invention. A feed additive for aquaculture of the present invention includes Bacillus amyloliquefaciens and postbiotics of Lactobacillus paracasei.

Preferably, the Feed additive for aquaculture further includes a fermented soybean powder. The fermented soybean powder is plant-based soybean powder or plant-based soybean powder fermented by microorganisms. In this embodiment, the fermented soybean powder is the plant-based soybean powder, which can replace fishmeal to reduce feed costs.

The Bacillus amyloliquefaciens and the postbiotics of Lactobacillus paracasei are mixed in a ratio of 1:10 based on counts of the Bacillus amyloliquefaciens and the Postbiotics of Lactobacillus paracasei. Specifically, a count of the Bacillus amyloliquefaciens is higher than and equal to 0.5×10⁹ cfu/g (colony-forming unit per gram). A count of the postbiotics of Lactobacillus paracasei is higher than and equal to 0.5×10¹⁰ cfu/g (colony-forming unit per gram). Preferably, the count of the Bacillus amyloliquefaciens is higher than and equal to 1.0×10⁹ cfu/g (colony-forming unit per gram). The count of postbiotics of Lactobacillus paracasei is higher than and equal to 1.0×10¹⁰ cfu/g (colony-forming unit per gram). Specifically, the count of Bacillus amyloliquefaciens is 4×10¹⁰ cfu/g (colony-forming unit per gram). The count of the postbiotics of Lactobacillus paracasei is 2×10¹⁰ cfu/g (colony-forming unit per gram).

In this embodiment, a weight ratio of the Bacillus amyloliquefaciens, the postbiotics of Lactobacillus paracasei and the fermented soybean powder is 1:20:19˜79, as shown in Table 1 below.

After feeding the feed additive for aquaculture of the present invention to aquatic animals, it can improve their feed conversion ratio (FCR), enhance their growth performance, increase their survival rate, boost their antibacterial capability, and reduce feed costs.

The feed additive is for crustaceans farmed in an aquaculture. Preferably, the crustaceans are shrimps, such as the white shrimps (Penaeus vannamei) and the whiteleg shrimps (Litopenaeus vannamei).

The feed additive for aquaculture further includes an excipient.

In this embodiment, a dosage form of a composition of the feed additive for aquaculture is a powder form or a liquid dosage form.

The postbiotics of Lactobacillus paracasei are heat-inactivated and includes heat-inactivated bacteria, bacterial fragments, and metabolites produced during the fermentation culture process.

A use of the feed additive for aquaculture mentioned above is for improving feed conversion ratio (FCR), which is further for improving the growth performance of the aquatic animals and increasing the survival rate of the aquatic animals and clearance efficiency of Vibrio. Specifically, the aquatic animals are shrimps, such as the white shrimps (Penaeus vannamei) and the whiteleg shrimps (Litopenaeus vannamei).

Specifically, the postbiotics refer to substances produced by probiotics that are beneficial to the host, or fragments of the bacterial cells.

The experimental feeds described below are groups A, B, C, and D in Table 1, with groups C and D being different combinations of bacterial counts of the present invention.

### [White Shrimp Growth Performance Test Method]

Use the whiteleg shrimps (Litopenaeus vannamei) with an initial weight of 1.63±0.02 g (grams), they were divided into five groups (including the control group and the groups of the present invention) (the experimental design for each group as shown in Table 1). Each group was tested in triplicate, with each replicate conducted in a fiberglass reinforced plastic (FRP) tank for the growth test. The water volume was 1 ton, with a total of 15 fiberglass reinforced plastic (FRP) tanks. Each tank contained 30 shrimp (the average weight between groups showed no statistical difference.).

The whiteleg shrimps (Litopenaeus vannamei) were fed 4% to 6% of experimental feed (groups A, B, C, and D in Table 1) daily. During the experiment, the water temperature was maintained at 28° C.±1.0° C., with a salinity of 20‰. The growth trial lasted for 28 days, and weight changes and feed intake of the whiteleg shrimps (Litopenaeus vannamei) were recorded on days 14 and 28. Additionally, the fiberglass reinforced plastic (FRP) tanks were cleaned, and the whiteleg shrimps (Litopenaeus vannamei) were weighed and counted at these intervals. The percent weight gain (PWG), the feed conversion ratio, and the survival rate were calculated according to the method described by Kuo et al. (2021). Table 2 below shows the counts of Bacillus amyloliquefaciens and the counts of postbiotics of Lactobacillus paracasei in groups A, B, C, and D.

TABLE 1

| | | Postbiotics of | | | |
|---|---|---|---|---|---|
| | | Lactobacillus | | | |
| | Bacillus | paracasei | | | |
| | amyloliquefaciens | (Lactobacillus | | | |
| | (Bacillus | paracasei | | | |
| | amyloliquifacience | L022) | | fermented | |
| | BA207) | (2 × 10\hat{ }10 | | soybean | Total |
| | (4 × 10\hat{ }10 cfu/g) | cfu/g) | Maltodextrin | powder | grams |
| groups | grams(g) | grams (g) | grams (g) | grams (g) | (g) |
| A | 2.5 | — | 97.5 | — | 100 |
| B | — | 50 | 50 | — | 100 |
| C | 2.5 | 50 | — | 47.5 | 100 |
| D | 1.25 | 25 | — | 73.75 | 100 |

TABLE 2
Counts for each group

| | Bacillus | Postbiotics of |
|---|---|---|
| | amyloliquefaciens | Lactobacillus |
| | (Bacillus | paracasei |
| | amyloliquifacience | (Lactobacillus paracasei |
| groups | BA207) | L022) |
| A | 1 × 10⁹ | — |
| | cfu/g | |
| B | — | 1 × 10¹⁰ |
| | | cfu/g |
| C | 1.0 × 10⁹ | 1 × 10¹⁰ |
| | cfu/g | cfu/g |
| D | 0.5 × 10⁹ | 0.5 × 10¹⁰ |
| | cfu/g | cfu/g |

The results are shown in FIG. 1. On days 14 and 28 of the experimental period, the percent weight gain (PWG) of groups C and D were higher than that of the other groups. On day 28, the percent weight gain (PWG) of group C exceeded 50%, significantly higher than the other experimental feed groups.

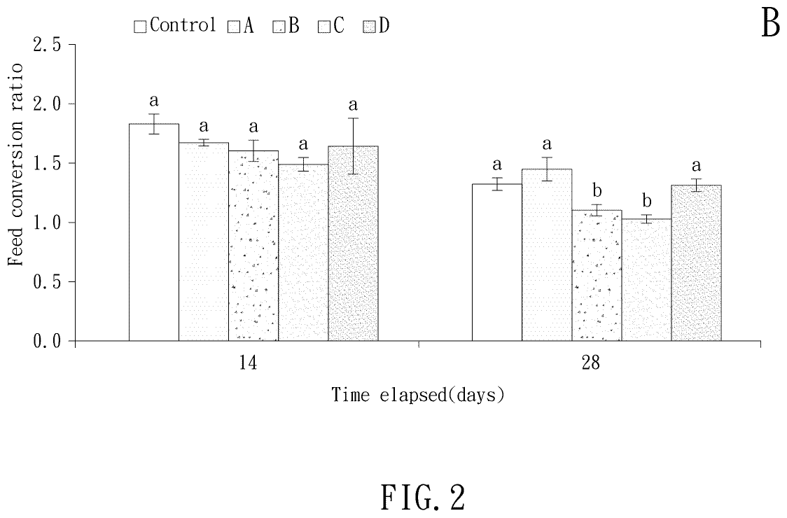

The feed conversion ratio (FCR) results are shown in FIG. 2. On day 14 of the experimental period, the feed conversion ratio of groups C and D were low. Additionally, on days 14 and 28, the feed conversion ratio of group C significantly decreased and was lower than that of the other experimental feed groups. Accordingly, from experimental feed groups C and D, it can be concluded that the present invention (the combination of the Bacillus amyloliquefaciens and the postbiotics of Lactobacillus paracasei) added to the feed can improve the feed conversion ratio (FCR) of the aquatic animals.

In the present invention, the Bacillus amyloliquefaciens is Bacillus amyloliquefaciens BA207, but it is not limited to this strain and can also be any commercially available Bacillus amyloliquefaciens. Similarly, the Lactobacillus paracasei is Lactobacillus paracasei L022, but it is not limited to this strain and can also be nay commercially available Lactobacillus paracasei.

[Selection of Bacillus amyloliquefaciens BA207]

Collect feces or samples from the target animals at the rearing site (from areas with better growth performance) using sterile bags, selecting feces with good form and avoiding those with diarrhea or loose stools. In the laboratory, take 1 gram of the feces or samples, add it to sterile water, and homogenize it thoroughly. Transfer 0.1 ml of the homogenate to LB broth (Lysogeny broth) for culture, and incubate at 37° C. for 24 hours, then perform serial dilutions to observe and culture the colonies. Pick a single colony and streak it on an LB (Lysogeny broth) agar plate for isolation to select probiotic strains. Add the isolated strains to 40% glycerol and store them in a −80° C. freezer.

### [Screening Antimicrobial Activity]

The screening steps are as follows:

Take a single colony of Escherichia coli (E. coli) (BCRC11509) and culture it in LB (Lysogeny broth) medium at 37° C. for 24 hours. Add 0.1% of the Escherichia coli (E. coli) (BCRC11509) culture to the cooled LB agar (Lysogeny agar, LB). Place an Oxford cup on the plate. Pour 20 ml of LB agar containing Escherichia coli (E. coli) (BCRC11509) onto the plate. Allow it to solidify before use. Remove the Oxford cup. Add 100 μl of probiotic culture to each well. Incubate at 37° C. for 20 hours. Measure the inhibition zones. Finally, select the strains that show the best performance in inhibiting Escherichia coli (E. coli) (BCRC11509) based on the size of the inhibition zones.

[Selection of Lactobacillus paracasei L022]

The screening steps are as follows:

Collect feces or samples from the target animals at the rearing site (from fields with better growth performance in the aquaculture farm) by using sterile bags, selecting feces with good form and avoiding those with diarrhea or loose stools. In the laboratory, take 1 gram of the feces or samples, add it to sterile water, and homogenize it thoroughly. Transfer 0.1 ml of the homogenate to MRS (deMan, Rogosa, and Sharpe) broth for culture and incubation at 37° C. for 24 hours. Then perform serial dilutions to observe the lactic acid bacteria colonies. Pick a single colony and streak it on an MRS agar plate for isolation to select lactic acid bacteria strains. Add the isolated strains to 40% glycerol and store them in a −80° C. freezer.

### Screening for Extracellular Polysaccharides:

1. Extraction method: Step 1. Heat bacterial solution and then centrifuge to collect the supernatant. Step 2. Take 5 mL of the sample and add it to a 50 mL centrifuge tube, then add an appropriate amount of ethanol and mix well. Step 3. Place the mixture in a 4° C. refrigerator for 24 hours. Step 4. Centrifuge the mixture at 9000 rpm and 4° C. for 10 minutes. Step 5. Discard the supernatant of the mixture; the precipitate at the bottom is the crude polysaccharide extract. Step 6. Redissolve the crude polysaccharide extract in 5 mL of RO water, and add an appropriate amount of ethanol and mix well. Repeat steps 2 to 4. Step 7. Directly freeze-dry the crude polysaccharide extract. Step 8. Dilute the extracted polysaccharides by 10-fold and 100-fold, and then determine their concentration by using the phenol-sulfuric acid method.
2. Phenol-sulfuric acid method

### Reagent Preparation Method:

Glucose Standard Solution: Prepare standard solutions with concentrations of 0.0025%, 0.0050%, and 0.0100%.

5% Phenol Solution: Weigh 5 g of phenol and dissolve it in deionized water, then quantify a total volume to 100 mL. Phenol needs to be placed in a 60° C. water bath to melt before weighing. Use 95% concentrated sulfuric acid.

Analysis Steps: Take 0.2 ml of the standard solution or sample dilution and add it to a 1.5 ml microcentrifuge tube; add 0.2 ml of 5% phenol solution and mix them well; add 1 ml of 95% concentrated sulfuric acid and mix them well. For this step, the 95% concentrated sulfuric acid should be added slowly along the tube wall of the 1.5 ml microcentrifuge tube, and note that it will release heat. Analyze the absorbance value at OD490 nm.

Finally, based on the results of the phenol-sulfuric acid method, select the strains with better extracellular polysaccharide content.

### [Preparation Method and Steps for Postbiotics]

Culture the lactic acid bacterial strain on MRS agar medium at an appropriate temperature for 24 to 48 hours. The MRS agar medium should be sterilized at 121° C. for 20 minutes before inoculation. Pick a single colony and inoculate it into an Erlenmeyer flask containing MRS broth and incubate statically at an appropriate temperature for 24 to 48 hours. The MRS broth in the Erlenmeyer flask should be sterilized at 121° C. for 20 minutes before inoculation. Add an appropriate culture medium such as glucose, yeast extract, soybean protein peptone, dipotassium phosphate, manganese sulfate, magnesium sulfate, and calcium chloride to the bioreactor. Sterilize the bioreactor by heating it with steam to 121° C. for 20 minutes. After completing the sterilization process, cool it down and adjust the temperature for production of postbiotics. Inoculate the culture from the Erlenmeyer flask containing the cultured lactic acid bacteria into the bioreactor and cultivate under appropriate pH control and aeration conditions with stirring to produce postbiotics. After the lactic acid bacteria have exhausted the glucose, perform postbiotic heat extraction at a high temperature of 100° C. for 30 minutes. After the liquid extraction is completed, add appropriate protectants (trehalose, maltodextrin, glycine, vitamin C) and excipients (e.g., soybean powder, calcium carbonate, and silicon dioxide). Mix and stir all the ingredients evenly until dissolved. Subject the liquid with added the protectants and the excipients to spray drying, with the inlet temperature of the hot air controlled between 120° C. and 170° C. and the outlet temperature controlled between 70° C. and 90° C.

### [White Shrimp Immunoassay Method]

On the 28th day after feeding, at each time point, hemolymph was sampled from two shrimp per repetition, with a total of six shrimps per group, for the detection of immune factors. On day 0, the hemolymph was sampled from six untreated shrimp for the detection of immune factors, serving as the initial control group. Hemolymph was drawn from the hemocoel of the cephalothorax of Litopenaeus vannamei using a sterile 1 mL syringe with a 25 G needle and was immediately mixed with an anticoagulant composed of 30 mM trisodium citrate, 0.34 M sodium chloride, and 10 mM ethylenediaminetetraacetic acid (EDTA), adjusted to pH 7.55, at a 1:10 ratio. Then, the mixture was placed on ice for subsequent experiments.

### (1). Total Haemocyte Count (THC)

Take 20 μL of the hemolymph-anticoagulant mixture and place it on a hemocytometer, and use a phase contrast microscope (Olympus CH40RF100, Tokyo, Japan) to count the total haemocyte count (THC).

### (2). Prophenoloxidase (PO)

Prophenoloxidase is activated by trypsin, a type of serine protease, through cleavage activation to produce phenoloxidase (PO) activity. L-DOPA (L-3, 4-dihydroxyphenylalanine, Sigma) is metabolized into dopachrome by the action of phenoloxidase (D-9628, Sigma, St. Louis, MO, USA). Take 1 mL of the hemolymph-anticoagulant mixture and centrifuge at 300×g for 15 minutes at 4° C., remove the supernatant, and add pre-chilled cacodylate-citrate buffer to wash the hemocytes. Repeat this washing process twice. Resuspend the hemocytes in 200 μL of cacodylate-citrate buffer, carefully mixing to avoid cell rupture. Take 100 μL of the suspension and add 50 μL of trypsin to induce the suspension at room temperature for 10 minutes. Then add 50 μL of L-DOPA (L-3, 4-dihydroxyphenylalanine, Sigma) and incubate in the dark at room temperature for 5 minutes. Add 800 μL of cacodylate-citrate buffer, and immediately measure the absorbance at OD490 nm using a Hitachi U-2001 spectrophotometer (Hitachi Ltd., Tokyo, Japan). The absorbance reflects the phenoloxidase (Prophenoloxidase, PO) activity in the hemocytes from 50 μL of hemolymph.

### (3) Lysozyme Activity

The lysozyme activity in plasma was measured using the methods of Ellis (1990) and Obach (1993). Dissolve Micrococcus luteus (0.2 mg/mL) in 200 μL of 0.05 M sodium phosphate buffer (pH 6.2), and add 10 μL of plasma to the mixture. Mix thoroughly and incubate at 27° C. Measure the absorbance at 530 nm using a continuous wavelength microplate spectrophotometer (SpectraMax® 190, Molecular Devices) at 30-second intervals for a total duration of 6 minutes. Finally, calculate the lysozyme concentration using a standard curve prepared with lysozyme standards.

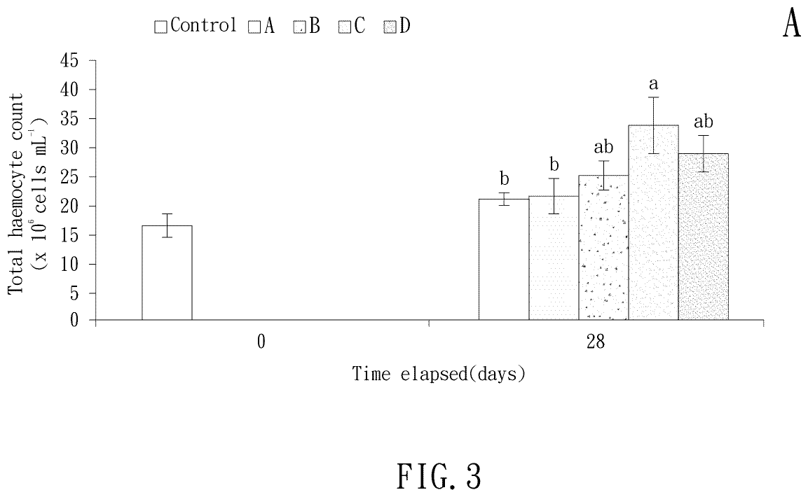

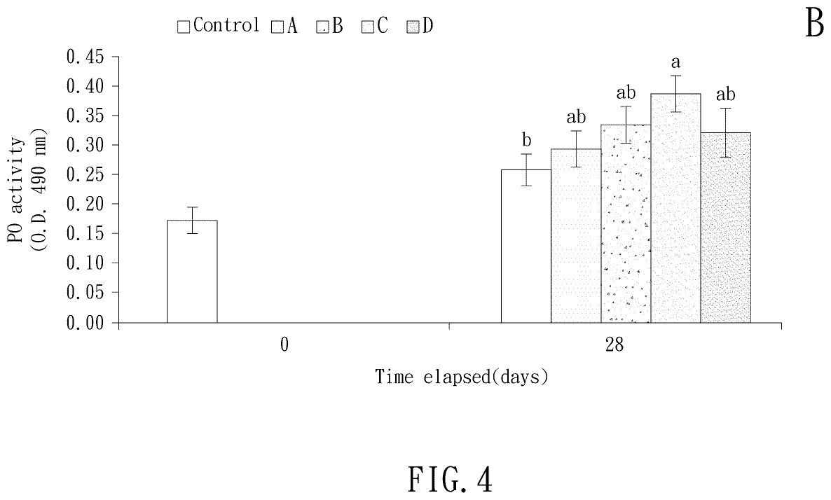

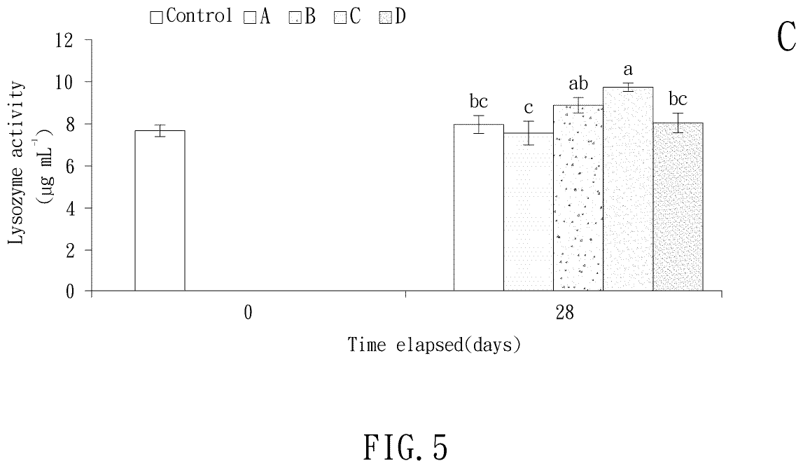

As shown in FIG. 3, on the 28th day after feeding, the total haemocyte count (THC) in experimental diet groups C and D was higher than in the other experimental diet groups. As shown in FIG. 4, the phenoloxidase (Prophenoloxidase, PO) activity in experimental diet groups C and D was higher, with group C being significantly higher than the other experimental groups. As shown in FIG. 5, on the 28th day, the lysozyme activity in experimental groups C and D was greater than 7 μg mL⁻¹, with group C being higher than the other experimental groups. Therefore, the feed additive for aquaculture of the invention, can enhance the immunity and increase the survival rate of aquatic species such as shrimp and white shrimp.

### [Vibrio Clearance Test Method]

Phagocytic activity and clearance efficiency were assessed on the 28th day of feeding. Five groups were tested, with three replicates per group and two shrimp per repetition. Each shrimp was injected with 20 μL of Vibrio alginolyticus (10\hat{ }9 cfu/mL), resulting in a final bacterial load of 2×10\hat{ }7 cfu/shrimp (colony-forming units/shrimp) in shrimp. After injection, the shrimp were transferred to 40 liters of seawater at a temperature of 28±1° C. and a salinity of 20‰. And after 60 minutes, 100 μL of hemolymph was extracted and added to 400 μL of anticoagulant, mixed thoroughly, and then used for phagocytic activity and clearance efficiency assays.

### (1). Phagocytic Activity

Phagocytic activity was assessed using the method described by Weeks-Perkins et al. (1995). A 200 μL mixture of hemolymph and anticoagulant was taken and mixed with 1% paraformaldehyde at a 1:1 ratio. The mixture was then reacted at 4° C. for 30 minutes to fix the hemocytes. It was centrifuged at 400×g for 10 minutes at 4° C., and the supernatant was removed. The hemocytes were resuspended in 0.85% NaCl saline solution. A 50 μL sample was taken and added to a microscope slide. The sample was centrifuged at 113×g for 3 minutes by a cytocentrifuge (Model Cytospin 3, Shandon, UK). After air drying, the sample was stained using Lius stain. Hemocytes were observed under a optical microscope, and those with stained bacteria inside were identified as phagocytic hemocytes. The phagocytic activity was determined by calculating the proportion of phagocytic hemocytes out of 200 hemocytes: Phagocytic activity (PA)=[(phagocytic hemocytes)/(total hemocytes)]×100% (Kuo et al. 2021).

### (2). Clearance Efficiency

Clearance efficiency was measured using the method of Adams (1991). A 100 μL mixture of hemolymph and anticoagulant was taken and diluted 5 times with 400 μL of 0.85% NaCl saline solution. Then, a 50 μL aliquot of the diluted mixture was spread onto tryptic soy agar (TSA, Bacto) containing 2% sodium chloride (NaCl) and using a disposable L-shaped plastic spreader. Each group was plated in triplicate and incubated at 28° C. for 16 hours. After incubation, colony-forming units (cfu) were counted to determine clearance efficiency. The formula used is as follows (Kuo et al. 2021): Clearance efficiency=[(cfu in test group)/(cfu in control group)]×100%.

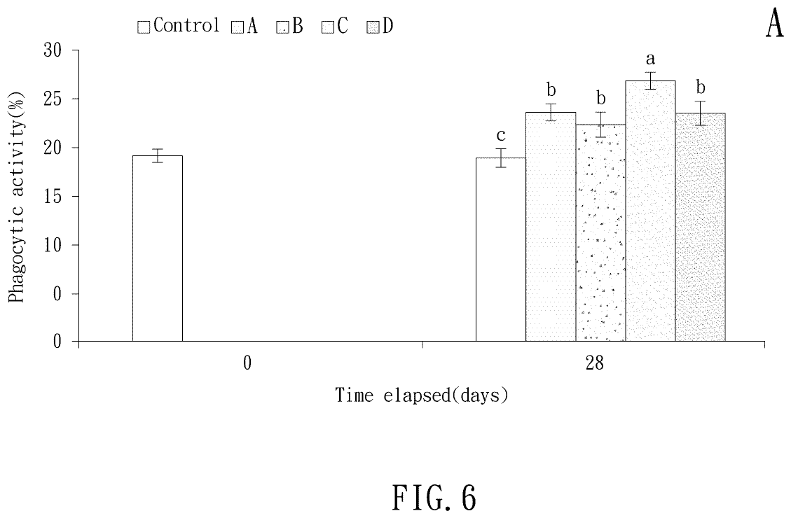

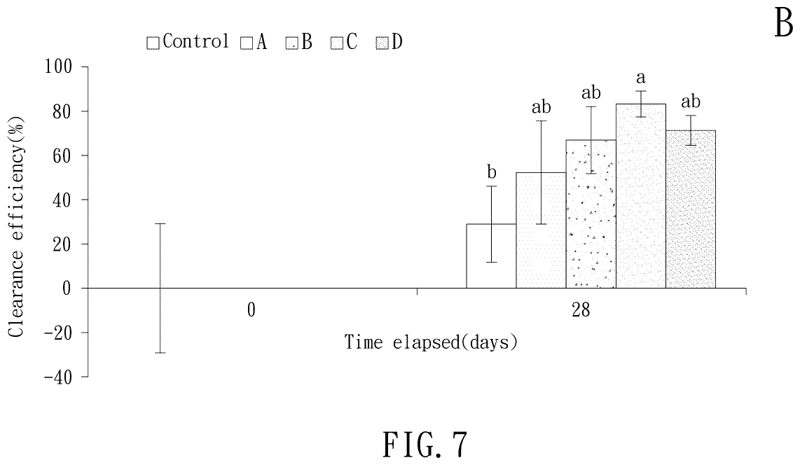

As shown in FIG. 6, on the 28th day of feeding, the phagocytic activity of experimental groups C and D was high. Furthermore, as shown in FIG. 7, the clearance efficiency of experimental groups C and D on the 28th day was higher than that of the other experimental groups. Therefore, the feed additive for aquaculture of the present invention can enhance the clearance efficiency of Vibrio and improve the anti-vibriosis ability, resulting in a high survival rate in aquatic animals.

### [Survival Assay Method for White Shrimp Post-Challenge]

The disease resistance was tested after feeding experimental diets (Groups A, B, C, D from Table 1, and a control group) for 21 days. There were six groups in total, each with three repetition, and 10 shrimp per repetition.

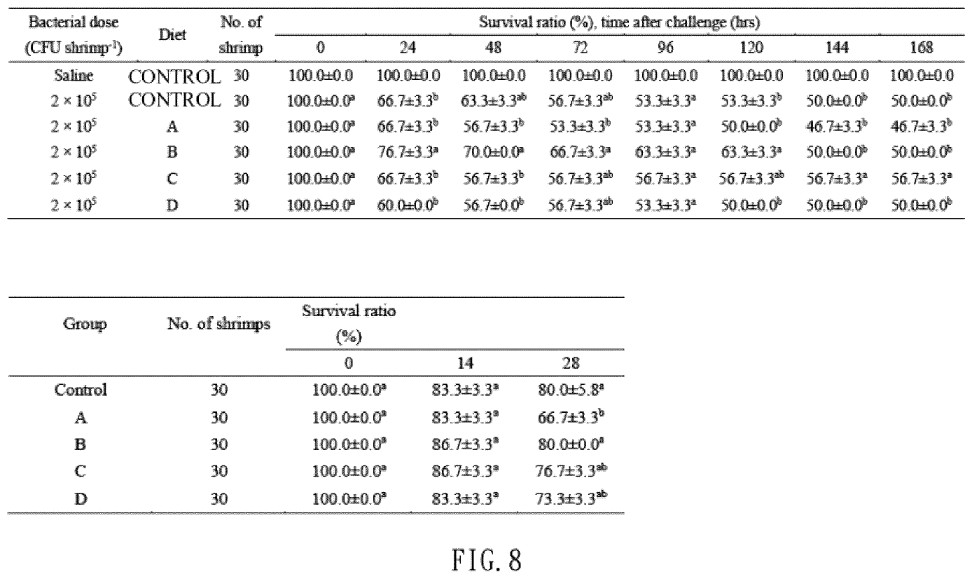

Each shrimp was injected with 20 μL of Vibrio alginolyticus (10∂cfu/mL), resulting in a final bacterial load of 2×10\hat{ }5 cfu/shrimp. After injection, the shrimp were transferred to 40 liters of seawater with 20‰ salinity at a temperature of 28±1° C. The shrimp were fed the experimental diets as usual in the challenge period. The numbers of surviving and dead shrimp were observed and recorded at 24, 48, 72, 96, 120, 144, and 168 hours post-challenge. Shrimp that died during the experiment were immediately removed and discarded after autoclaving. As shown in FIG. 8, at 168 hours after challenge, the survival rates of experimental groups C and D were high.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A feed additive for aquaculture, including Bacillus amyloliquefaciens and postbiotics of Lactobacillus paracasei.

2. The feed additive for aquaculture of claim 1, further including a fermented soybean powder.

3. The feed additive for aquaculture of claim 1, wherein a count of the Bacillus amyloliquefaciens is higher than and equal to 0.5×10⁹ cfu/g (colony-forming unit per gram).

4. The feed additive for aquaculture of claim 1, wherein a count of the postbiotics of Lactobacillus paracasei is higher than and equal to 0.5×10¹⁰ cfu/g (colony-forming unit per gram).

5. The feed additive for aquaculture of claim 1, wherein the Bacillus amyloliquefaciens and the postbiotics of Lactobacillus paracasei are mixed in a ratio of 1:10 based on counts of the Bacillus amyloliquefaciens and the postbiotics of Lactobacillus paracasei.

6. The feed additive for aquaculture of claim 1, wherein the feed additive is for crustaceans farmed in an aquaculture.

7. The feed additive for aquaculture of claim 2, wherein a weight ratio of the Bacillus amyloliquefaciens, the postbiotics of Lactobacillus paracasei and the fermented soybean powder is 1:20:19˜79; a count of the Bacillus amyloliquefaciens is higher than and equal to 1.0×10⁹ cfu/g (colony-forming unit per gram); a count of postbiotics of Lactobacillus paracasei is higher than and equal to 1.0×10¹⁰ cfu/g.

8. The feed additive for aquaculture of claim 7, wherein the postbiotics of Lactobacillus paracasei are heat-inactivated and includes heat-inactivated bacteria, bacterial fragments, and metabolites produced during the fermentation culture process; the fermented soybean powder is plant-based soybean powder or plant-based soybean powder fermented by microorganisms; the feed additive for aquaculture further includes an excipient; a dosage form of a composition of the feed additive for aquaculture is a powder form or a liquid dosage form; the feed additive is for crustaceans farmed in an aquaculture; the crustaceans are shrimps.

9. A use of the feed additive for aquaculture of claim 1 for improving feed conversion ratio (FCR).

10. The use of the feed additive for aquaculture of claim 9 for improving a growth performance of aquatic animals and increasing a survival rate of the aquatic animals and clearance efficiency of Vibrio; and the aquatic animals are shrimps.
